(19) United States
(12) Patent Application Publication
(10) Pub. No.: US 2023/0009798 A1
(43) Pub. Date: Jan. 12, 2023

(54) SILICONE IMPLANT ADJUNCT FORMULATIONS AND METHODS OF USE

(71) Applicant: Kayte R. Susse, Los Angeles, CA (US)

(72) Inventor: Kayte R. Susse, Los Angeles, CA (US)

(21) Appl. No.: 17/831,670

(22) Filed: Jun. 3, 2022

Related U.S. Application Data
(60) Provisional application No. 63/219,677, filed on Jul. 8, 2021.
(60) Provisional application No. 63/219,590, filed on Jul. 8, 2021.

(51) Int. Cl. A23L 33/15; A61K 31/375; A61K 31/07; A61K 31/198; A61K 31/355; A61K 31/714; A61K 33/30; A61K 33/06; A61K 31/122; A61K 31/519; A23L 33/105

(52) U.S. Cl. CPC A23L 33/15; A61K 31/375; A61K 31/07; A61K 31/198; A61K 31/355; A61K 31/714; A61K 33/30; A61K 33/06; A61K 31/122; A61K 31/519; A23L 33/105

(57) ABSTRACT

A nutritional supplement product may be formulated to be used or digested by a person and to support the prevention of systemic health concerns that can arise after implanting exogenous silicone devices inside the human body. Such supplements may also promote the healthy production and balance of scar tissue, including a scar tissue capsule. Such supplement may be used as an adjunct therapy to a silicone device implantation procedure.

### BACKGROUND

### 1. Related Applications

This utility patent application claims the benefit of U.S. Provisional patent Application Ser. No. 63/219,590, filed on Jul. 8, 2021, and U.S. Provisional patent application Ser. No. 63/219,677, filed on Jul. 8, 2021; all of which are hereby incorporated by this reference in their entireties.

### 2. Field of the Invention

This invention relates to dietary, nutritional supplements and investigational peptides, namely, nutritional supplements and amino acid peptides used to promote the healthy formation of scar tissue, including the healthy formation of a scar tissue capsule.

### 3. The Background Art

Nutritional supplements come in a variety of forms and may be used for a variety of purposes. A nutritional supplement may take the form of a pill, capsule, tablet, powder, gel, or liquid. Generally, a nutritional supplement provides a direct source of one or more nutrients or compounds that are intended to support a specific purpose. A nutritional supplement may include natural and/or synthetic components.

Adjunct therapy, or adjuvant therapy, is a therapy or treatment given in addition to a primary therapy, or initial therapy. The adjunct therapy is given to assist the primary therapy, or help maximize the effectiveness of the primary therapy. The use of adjunct therapy to help in certain circumstances is known, perhaps most prevalently in association with cancer treatments. However, no adjunct therapy has been developed specifically for individuals that have undergone silicone device implant procedures, such as breast implant procedures and the like.

A person may undergo breast implant surgery for a variety of reasons. Breast implants may be implanted to restore a natural looking breast following a mastectomy, to correct congenital defects and deformities of the chest wall, or to alter the appearance of the breast for cosmetic purposes. Breast implants are generally categorized based on their contents, or filler material. A saline implant may have an elastomer silicone shell that may be filled with a sterile saline solution during surgery. A silicone implant may have an elastomer silicone shell that is filled with a viscous silicone gel before surgical implantation. A structured implant may use nested elastomer silicone shells and two saline filled lumen. Largely discontinued, a composite implant may feature miscellaneous fillers, i.e., soy oil or polypropylene string.

One of the complications with breast implants is that they can rupture, or can begin to leak their contents into the body after implantation. However, an implant does not have to rupture to cause physical problems to a person that has an implant. Another possible complication that can result from a silicone implant is “implant gel bleed,” which can also be described as a silicone implant “sweating” inside the body after implantation. All implants “sweat” differently inside the body due to reactions of the immune system, and other factors.

Breast implants are just one type of silicone implant. Other types of silicone implants may include, butt implants, chin implants, jaw implants, pectoralis implants, calf implants, penile implants, and more. The complications associated most commonly with silicone breast implants can occur with any type of silicone implant.

A growing number of individuals report an array of symptoms or complications at all life stages of having silicone implants, or silicone devices, in their bodies. Such symptoms may arise from early implantation to many years post implantation. Such symptoms have been the subject of ongoing, recent investigations with the FDA and the subject of changes to state laws related to informed consent and implant safety, including the possibility of developing various systemic illnesses. These emerging health concerns are in addition to existing health concerns related to the risks of surgery.

What is needed, and currently missing, is a nutritional supplement therapeutic that may be used as an adjunct, or adjunct therapy, to silicone implant procedures. Such an adjunct nutritional supplement may support the body with consideration to the development of key disease processes resultant from the implantation of silicone devices, thereby fostering and supporting a healthy and satisfying experience with silicone implants in the body.

What is needed is a nutritional supplement and therapeutic specifically designed to promote the formation of healthy scar tissue. This may include the formation of a scar tissue capsule, which may form around foreign components within a human body, including but not limited to, silicone implants.

### SUMMARY OF THE INVENTION

In view of the foregoing, certain embodiments of a product, compound and method in accordance with the invention provide a nutritional supplement, or an oral product, that can be consumed by a person to facilitate and promote the healthy production of scar tissue, including a scar tissue capsule. The nutritional supplement, or oral product, may be suitably formulated to promote production of healthy scar tissue in humans. The nutritional supplement, or oral product, may be used as an adjunct with silicone implant surgeries.

The nutritional supplement, or oral product, may be used by any person who has a silicone implant device inside their body, regardless of the type, shape, location, or age of the implant device. The nutritional supplement, or oral product, is intended to be used for the entire time a person has a silicone implant device insider their body, i.e., weekly use, daily use, twice daily use, or the like. The nutritional supplement, or oral product, may be taken orally with or without food any time of day.

The nutritional supplement, or oral product, is intended to be used as a supportive therapeutic. No current product or formulations aim to address the problems and complications associated with silicone implants once inside the body in a preventative manner. Preventing the systemic issues which can arise may prevent the need for costly and traumatic explant surgery, or removal surgery for the implants, which is currently the standard response as problems or complications develop from having a silicone implant inside the body.

In certain embodiments, a daily nutritional supplement formulation supports the prevention of several key systemic health concerns that can arise after implanting exogenous silicone devices inside a human body. The supplement may be taken daily as an adjunct the entire time a person has silicone implants. The nutritional supplement may be taken or used as directed or recommended by a doctor.

In certain embodiments, a nutritional supplement, or oral product, may be formulated to support certain MMP pathways. Matrix metalloproteinases (MMPs) belong to the metzincin family of zinc-containing multi-domain molecules, and can act as soluble membrane-bound proteases. MMPs degrade extracellular matrix components and thus facilitate tissue degradation and subsequent remodeling. In particular, MMP-9 may primarily have a profibrotic effect.

In certain embodiments, the oral product may be formulated to be a powder, which may be included in a pill or capsule. In a separate embodiment, the oral product may be formulated to be a liquid that is ready for consumption. In a separate embodiment, the oral product may be formulated to be a gel or gum that is ready for consumption. Such separate embodiments may be considered a dose of the respective embodiment.

In certain embodiments, the supplement formulations may include one or more individual supplement formulations, which can be taken together or separately, and may be interchanged or combined depending on a desired outcome.

In a separate embodiment, the oral product may be formulated to be a food product that is ready for consumption. In a separate embodiment, the product may be administered by a variety of methods, including without limitation, orally, subcutaneously, intravenously, and intramuscularly.

In one embodiment, a method for promoting health while a silicone implant, or silicone device, is in a human body may comprise the following steps: providing a nutritional supplement that may be considered an adjunct therapy to a silicone device implantation procedure, which procedure implants a silicone implant, or silicone device, in a human body; and ingesting the nutritional supplement while the silicone implant, or silicone device, is in the human body. The nutritional supplement used as an adjunct therapy may be ingested at least once a day, or in accordance with a doctor's recommendations, while the silicone implant, or silicone device, is in the human body.

The nutritional supplement used as an adjunct therapy may comprise a base component and a collagen support blend. The base component may comprise an effective amount of Vitamin A, an effective amount of Vitamin C, an effective amount of Vitamin D, an effective amount of Vitamin E, an effective amount of Vitamin K2, an effective amount of Folate, an effective amount of Vitamin B12, an effective amount of Calcium, an effective amount of Zinc, and an effective amount of Selenium.

The collagen support blend may comprise an effective amount of L-Lysine HCl, an effective amount of Bromelain, an effective amount of L-Proline, an effective amount of Quercetin, an effective amount of L-Carnosine, and effective amount of Centella asiatica Extract, and effective amount of Aloe vera Extract, an effective amount of Zinc Picolinate, and an effective amount of KVP as Lysine-Proline-Valine (L-Lysyl—L-valyl—L-proline).

The nutritional supplement used as an adjunct therapy may further comprise an immune modulation blend. The immune modulation blend may comprise an effective amount of L-Glutamine, an effective amount of Mixed Tocopherols (a mixture of multiple different types of Vitamin E), an effective amount of Glycine, an effective amount of PQQ (pyrroloquinoline quinone), and an effective amount of Cholecalciferol.

The nutritional supplement used as an adjunct therapy may further comprise a detox blend. The detox blend may comprise an effective amount of Carrot Root Extract, an effective amount of L-Leucine, an effective amount of Molybdenum Glycinate, an effective amount of Fulvic Acid, an effective amount of 5-methyltetrahydrofolate (5-MTHF), an effective amount of Calcium, and an effective amount of Methylcobalamin.

The nutritional supplement used as an adjunct therapy should not contain any heavy metals. For example and not by way of limitation, the nutritional supplement should not contain antimony, arsenic, barium, cadmium, chromium, lead, silicon, and/or mercury.

The nutritional supplement used as an adjunct therapy may support the balanced formation and/or growth of scar tissue near, adjacent to, and/or around a silicone implant, or silicone device, that is implanted inside a human body.

The nutritional supplement used as an adjunct therapy may dampen, or suppress, at least one of the MMP (matrix metalloproteinases) pathways of the human body. For example and not by way of limitation, the nutritional supplement may dampen, or suppress, an MMP pathway by decreasing the production of MMP-9.

### DETAILED DESCRIPTION OF THE ILLUSTRATED EMBODIMENTS

It will be readily understood that the components of the present invention, as generally described herein, could be arranged and designed in a wide variety of different configurations or formulations. Thus, the following more detailed description of the embodiments of the system, product and method of the present invention is not intended to limit the scope of the invention, as claimed, but is merely representative of various embodiments of the invention.

A nutritional supplement in accordance with the present invention may be comprised of multiple components, for example and not by way of limitation, nutrients, natural peptides, synthetic peptides, investigational peptides, vitamins, minerals, plant extracts, and the like. A nutritional supplement may include one or more of these components depending on the intended use. Thus, a nutritional supplement may include some but not all of the components, and may still be suitable for its intended purpose, i.e., an adjunct supportive therapy, or the like.

In one embodiment, the method of preparation, formulation, or chemical synthesis of healthy scar tissue is accomplished by using reactants and/or nutrients that include at least the following: ascorbic acid, glutamine (L), mixed tocopherols, glycine, vitamin K2 (as menaquinone 7, Mk7), selenomethionine, Pyrroloquinoline quinone, cholecalciferol, retinyl palmitate, and KVP as Lys-Pro-Val. Each of the components/reactants has a respective function, but together may have a separate function.

In one embodiment, a nutritional supplement may include a base composition, which base composition may be comprised of the following: 21 mcg RAE of Vitamin A (Retinyl Palmitate); 335 mg of Vitamin C (Ascorbic Acid-Non Corn); 17.5 mcg of Vitamin D (Cholecalciferol); 67 mg of Vitamin E (Mixed Tocopherols); 70 mcg of Vitamin K2 MK7; 200 mcg DFE of Folate (5-MTHF); 100 mcg Vitamin B12 (Methylcobalamin); 6 mg of Calcium (D-Glucarate); 700 mcg of Zinc (Picolinate); and 17 mcg of Selenium (L-Selenomethionine).

In the same embodiment, or another embodiment, 900 mg of an immune modulation blend may be included, which immune modulation blend may be comprised of the following: Ascorbic Acid, L-Glutamine, Mixed Tocopherols, Glycine, Vitamin K2 MK7, L-Selenomethionine, PQQ (pyrroloquinoline quinone), Cholecalciferol, and Retinyl Palmitate.

In the same embodiment, or another embodiment, 360 mg of a collagen response blend may be included, which collagen response blend may be comprised of the following: L-Lysine, HCl, Bromelain, L-Proline, Quercetin, L-Carnosine, Centella asiatica Extract, Aloe vera Extract, Zinc Picolinate, and Lysine/Proline/Valine.

In the same embodiment, or another embodiment, 210 mg of a gentle detox support blend may be included, which gentle detox support blend may be comprised of the following: Carrot Root Extract, Calcium D-Glucarate, L-Leucine, Molybdenum Glycinate, Fulvic Acid, 5-MTHF, Calcium, and Methylcobalamin.

In the same embodiment, or another embodiment, other ingredients may include cellulose and vegetable stearate. The base composition, immune modulation blend, collagen response blend, and gentle detox support blend may be described as one serving of a nutritional supplement in accordance with the invention. The amounts of the respective formulation components may vary within a range of approximately ±10% by weight.

In one embodiment, a capsule for a nutritional supplement may be comprised of purple carrots, which creates a plant-based capsule that provides a source of beta-carotene.

A nutritional supplement in accordance with the present invention should be taken as directed by a health care professional. It may be helpful to increase the amount of liquids a person drinks each day while taking the nutritional supplement.

In one embodiment, an immune modulation blend may be comprised of the following: 500-1000 mg of Ascorbic Acid; 500-100 mg of Glutamine (L); 0.1 mg of Mixed Tocopherols; 100 mg of Glycine; 0.1 mg of Vitamin K2, Mk7; 0.025 iu of Selenomethionine; 2.0 mg of PQQ; 1000-3000 iu of Cholecalciferol; 100 iu of Retinol Palmitate; and 50-500 mcg of KVP as Lys-Pro-Val.

An immune modulation blend, or any similarly formulated immune support supplement, provides the human body with nutrients that have been clinically researched to be missing in patients with an autoimmune disease process in the presence of exogenous foreign devices, i.e., silicone implants. Such a supplement utilizes key nutrients to support those with predisposition to the development of these processes in relationship to decreasing gut inflammation, provide substrates that promote and recycle glutathione, and foster immune modulation. The amounts of the respective formulation components may vary within a range of approximately ±10% by weight.

In one embodiment, a collagen response blend, or scar tissue response blend, may comprise the following: 100-500 mg of Lysine HCL; 100-500 mg of Bromelain; 100-500 mg of Proline; 100-500 mg of Quercetin; 50-300 mg of Carnosine; 46-500 mg of Centella Asiatica Extract; 46-500 mg of Aloe Vera Extract; 50-500 mcg of KVP as Lys-Pro-Val; and 1-5 mg of Zinc Picolinate.

A collagen response blend, or any similarly formulated collagen support supplement, supports the formation of a healthy collagen response of scar tissue capsule around an implant to ensure there is not too little scar tissue, primarily by accounting for the connection between various types of hypermobility, both generic and acquired, which may cause a maladaptive collagen response. Medically stated, a collagen response blend aims to improve collagen response in humans with silicone implants by supporting the MMP pathways. MMP pathways are a class of enzymes that belong to the zinc-metalloproteinases involved in the degradation of the extracellular matrix. A collagen response blend may primarily focus on MMP-9 and its role in histamine regulation and its relationship to skin laxity. A collagen response blend, especially including an effective amount of KVP as Lys-Pro-Val, may support the formation of a healthy scar tissue response in the presence of silicone implants such that the development of keloid or hyperactive scar tissue is avoided. Additionally, a collagen response blend, especially including an effective amount of KVP as Lys-Pro-Val, may improve wound healing in post-surgical wounds and/or scars. The amounts of the respective formulation components may vary within a range of approximately ±10% by weight.

In one embodiment, a gentle detox support blend, or detoxification support blend, may comprise the following: 200-500 mg of Carrot root; 100-300 iu of Retinyl Palmitate; 100-500 mg Calcium d-glucarate; 26-300 mg of Leucine; 300 mg of Molybdenum chelate; 0.3 mg of Methylfolate; and 0.15 mg of Methylcobalamin.

A gentle detox blend, or any similarly formulated detox support supplement, may support vital pathways in the body responsible for detoxification in the presence of exogenous silicone implants with the primary goal of reducing reabsorption of exogenous estrogen-like compounds, or “xenoestrogens,” and correcting for genetic mutations in common methylation enzymes such as Methylene tetrahydrofolate reductase (MTHFR) and variants to support the body's detoxification process phase 1 and 2 as an adjunct to implantation with silicone devices. Additionally, preliminary studies also show the combination of retinoids and glucarate to be preventative in development of breast cancer, based on the relationship between implants and BIALCL (breast implant associated large cell lymphoma). The amounts of the respective formulation components may vary within a range of approximately ±10% by weight.

It may be noted that two components or nutrients, Vitamin A and KVP as Lys-Pro-Val (synthetic peptide), are repeated in various blends. This indicates the multi-faceted purposes of these components. In a single-formulation embodiment, this does not mean that the amount of these components should be doubled or tripled, but the stated quantity in each listing may reflect the quantity listed in a single-formulation embodiment.

A person undergoing a silicone implantation procedure may expect various, possible issues associated with having a silicone implant in their body. A nutritional supplement in accordance with the present invention may be designed or formulated to address one or more of such issues. For example and not by way of limitation, a person may experience issues related to the development of collagen, possible autoimmune reactions, and the presence of toxins.

Chronic toxins, i.e., chemicals and heavy metals, allergens, and pathogens that affect a person or patient prior to implantation of a silicone implant may drive upregulation of the MMP-9 pathway of the immune system, which may be described as one form of an “allergic response.” MMP-9 causes mast cell activation. Mast cell activation and priming drives excess histamine production. Excess histamine production degrades the collagen matrix in the body, specifically type IV collagen (Co14), but potentially others. Dysregulation of systemic collagen in the body by excess histamine in the body hinders the patient's ability to build a protective, productive capsule of collagen around their silicone implant after implantation. Dampening the degradation of collagen protects the patient's body from too thin a capsule tissue or too thick a capsule caused by maladaptive response, which leaves it vulnerable to the immune system's macrophages, which mobilize micro silicone and its chemical formula companions by phagocytosis for example.

In one embodiment, particularly an embodiment of a collagen response blend, a nutritional supplement may comprise effective amounts of quercitin, carnosine, Centella asiatica, Aloe vera, zinc, bromelain, lysine, proline and KVP as Lys-Pro-Val. This combination of nutrients is used to dampen the MMP-9 pathway, thereby reducing excess histamine and promoting the formation of healthy collagen, including collagen adjacent to and around a silicone implant device.

In the presence of new, foreign devices, and especially mobilized micro silicone, a patient may develop an autoimmune reaction. Such a reaction has been documented in silicone research.

In one embodiment, particularly an embodiment of an immune modulation blend, a nutritional supplement may comprise effective amounts of Vitamin C, glutamine, Vitamin E, Vitamin K, glysine, selenium, Vitamin D, Vitamin A, and PQQ.

The introduction of a silicone implant into the human body may also result in the introduction of certain toxins into the body. For example, xenoestrogens and other chemicals found in silicone devices, and in the environment, may hinder the health of a person with a silicone implant device.

In one embodiment, particularly an embodiment of a detox support blend, a nutritional supplement may comprise effective amounts of Vitamin A, calcium d-glucarate, leucine, molybdenum, methylfolate, methylcobalamin, and fulvic acid.

It is important that any nutritional supplement embodiment in accordance with the present invention not contain heavy metals, nor microbial contamination. One of the problems with implant rupture, and/or implant sweating, is the introduction of heavy metals into the body. A nutritional supplement in accordance with the present invention should not contribute to this problem.

In one embodiment, a method for promoting the health of a person while a silicone implant is inside the person's body may comprise the following steps: providing a nutritional supplement that is an adjunct therapy to a silicone implantation procedure, which procedure places, or implants, a silicone implant, or silicone implant device, in a human body; and ingesting the nutritional supplement. The nutritional supplement used as an adjunct therapy may comprise, or consist essentially of, or consist of, the following nutrients: about 300 mg of Quercetin, about 200 mg of Carnosine, about 200 mg of Centella asiatica extract, about 200 mg of Aloe vera extract, about 3 mg of Zinc, about 300 mg of Bromelain, about 300 mg of Lysine about 300 mg of Proline, and about 300 mg of KVP as Lysine-Proline-Valine.

The nutritional supplement used as an adjunct therapy may further comprise, or further consist essentially of, or further consist of, the following nutrients: about 750 mg of Ascorbic Acid, about 750 mg of Glutamine (L), about 0.1 mg of Mixed Tocopherols, about 100 mg of Glycine, about 0.1 mg of Vitamin K2 as menaquinone 7, about 0.025 iu of Selenomethionine (L-selenomethionine), about 2.0 mg of PPQ, about 2000 iu of Cholecalciferol, and about 100 iu of Retinyl Palmitate.

The nutritional supplement used as an adjunct therapy may further comprise, or further consist essentially of, or further consist of, the following nutrients: about 350 mg of Carrot Root Extract, about 100 iu of Retinyl Palmitate, about 350 mg of Calcium d-glucarate, about 150 mg of Leucine, about 300 mg of Molybdenum chelate, about 0.3 mg of Methylfolate, and about 0.15 mg of Methylcobalamin.

In one embodiment, a method for promoting the health of a person while a silicone implant, or silicone device, is inside a human body of the person may comprise the following steps: providing a nutritional supplement that may comprise about 21 mcg RAE of Vitamin A, about 335 mg of Vitamin C, about 17.5 mcg of Vitamin D, about 67 mg of Vitamin E, about 70 mcg of Vitamin K2, about 200 mcg DFE of Folate, about 100 mcg of Vitamin B12, about 6 mg of Calcium, about 700 mcg of Zinc, and about 17 mcg of Selenium; and ingesting the nutritional supplement at least once a day, or in accordance with a doctor's recommendation.

The nutritional supplement may further comprise a collagen support blend, an immune modulation blend, and/or a detox blend. The nutritional supplement should not contain or include heavy metals that are adverse to the intended use of the nutritional supplement. The nutritional supplement may be used to promote and/or support the healthy production of scar tissue, which scar tissue may form near, adjacent to, and/or around a silicone implant, or silicone implant device.

The collagen support blend may further comprise, or may further consist essentially of, or may further consist of, the following components: about 300 mg of Quercetin, about 200 mg of Carnosine, about 200 mg of Centella asiatica extract, about 200 mg of Aloe vera extract, about 3 mg of Zinc, about 300 mg of Bromelain, about 300 mg of Lysine, about 300 mg of Proline, and about 300 mg of KVP as Lys-Pro-Val.

The immune modulation blend may further comprise, or may further consist essentially of, or may further consist of, the following components: about 750 mg of Ascorbic Acid, about 750 mg of Glutamine (L), about 0.1 mg of Mixed Tocopherols, about 100 mg of Glycine, about 0.1 mg of Vitamin K2 as menaquinone 7, about 0.025 iu of Selenomethionine, about 2.0 mg of PQQ, about 2000 iu of Cholecalciferol, about 100 iu of Retinyl Palmitate, and about 300 mcg of KVP as Lys-Pro-Val.

The detox blend may further comprise, or may further consist essentially of, or may further consist of, the following components: about 350 mg of Carrot Root Extract, about 200 iu of Retinyl Palmitate, about 350 mg of Calcium d-glucarate, about 150 mg of Leucine, about 300 mg of Molybdenum chelate, about 0.3 mg of Methylfolate, and about 0.15 mg of Methylcobalamin.

The present invention may be embodied in other specific forms without departing from its fundamental functions or essential characteristics. The described embodiments are to be considered in all respects only as illustrative, and not restrictive. All changes which come within the meaning and range of equivalency of the illustrative embodiments are to be embraced within their scope.

What is claimed and desired to be secured by United States Letters Patent is:

1. A method for promoting health while a silicone implant is in a human body, comprising:
providing a nutritional supplement that is an adjunct therapy to a silicone implantation procedure that implants a silicone implant in a human body; and
ingesting the nutritional supplement while the silicone implant is in the human body.

2. The method of claim 1, wherein the nutritional supplement comprises a base component and a collagen support blend, wherein the base component comprises an effective amount of Vitamin A, an effective amount of Vitamin C, an effective amount of Vitamin D, an effective amount of Vitamin E, an effective amount of Vitamin K2, an effective amount of Folate, an effective amount of Vitamin B12, an effective amount of Calcium, an effective amount of Zinc, and an effective amount of Selenium, and the collagen support blend comprises an effective amount of L-Lysine HCl, an effective amount of Bromelain, an effective amount of L-Proline, an effective amount of Quercetin, an effective amount of L-Carnosine, an effective amount of Centella asiatica Extract, an effective amount of Aloe vera Extract, an effective amount of Zinc Picolinate, and an effective amount of Lysine-Proline-Valine.

3. The method of claim 2, wherein the nutritional supplement further comprises an immune modulation blend comprising an effective amount of L-Glutamine, an effective amount of Mixed Tocopherols, an effective amount of Glycine, an effective amount of PQQ, and an effective amount of Cholecalciferol.

4. The method of claim 3, wherein the nutritional supplement further comprises a detox blend comprising an effective amount of Carrot Root Extract, an effective amount of L-Leucine, an effective amount of Molybdenum Glycinate, an effective amount of Fulvic Acid, an effective amount of 5-MTHF, an effective amount of Calcium, and an effective amount of Methylcobalamin.

5. The method of claim 4, wherein the nutritional supplement does not contain heavy metals.

6. The method of claim 5, further comprising:
supporting the growth of scar tissue around the silicone implant.

7. The method of claim 5, wherein the nutritional supplement is ingested at least once a day while the silicone implant is in the human body.

8. The method of claim 6, further comprising:
dampening at least one of MMP pathways of the human body.

9. The method of claim 8, wherein the MMP pathway is dampened by decreased production of MMP-9.

10. A method for promoting health while a silicone implant is in a human body, comprising:
providing a nutritional supplement that is an adjunct to a silicone implantation procedure that places a silicone implant in a human body, wherein the nutritional supplement consists essentially of about 300 mg of Quercetin, about 200 mg of Carnosine, about 200 mg of Centella asiatica extract, about 200 mg of Aloe vera extract, about 3 mg of Zinc, about 300 mg of Bromelain, about 300 mg of Lysine, about 300 mg of Proline, and about 300 mg of KVP as Lys-Pro-Val; and
ingesting the nutritional supplement.

11. The method of claim 10, wherein the nutritional supplement further comprises about 750 mg of Ascorbic Acid, about 750 mg of Glutamine (L), about 0.1 mg of Mixed Tocopherols, about 100 mg of Glycine, about 0.1 mg of Vitamin K2 as menaquinone 7, about 0.025 iu of Selenomethionine, about 2.0 mg of PQQ, about 2000 iu of Cholecalciferol, and about 100 iu of Retinyl Palmitate.

12. The method of claim 11, wherein the nutritional supplement further comprises about 350 mg of Carrot Root, about 100 iu of Retinyl Palmitate, about 350 mg of Calcium d-glucarate, about 150 mg of Leucine, about 300 mg of Molybdenum chelate, about 0.3 mg of Methylfolate, and about 0.15 mg of Methylcobalamin.

13. The method of claim 12, wherein the nutritional supplement does not contain heavy metals.

14. A method for promoting health while a silicone implant is in a human body, comprising:
providing a nutritional supplement that is an adjunct to a silicone implantation procedure, wherein the nutritional supplement comprises about 21 mcg RAE of Vitamin A, about 335 mg of Vitamin C, about 17.5 mcg of Vitamin D, about 67 mg of Vitamin E, about 70 mcg of Vitamin K2, about 200 mcg DFE of Folate, about 100 mcg of Vitamin B12, about 6 mg of Calcium, about 700 mcg of Zinc, and about 17 mcg of Selenium; and
ingesting the nutritional supplement at least once a day.

15. The method of claim 14, wherein the nutritional supplement further comprises a collagen support blend, an immune modulation blend, and a detox blend.

16. The method of claim 15, wherein the nutritional supplement does not contain heavy metals.

17. The method of claim 15, wherein the collagen support blend comprises about 300 mg of Quercetin, about 200 mg of Carnosine, about 200 mg of Centella asiatica extract, about 200 mg of Aloe vera extract, about 3 mg of Zinc, about 300 mg of Bromelain, about 300 mg of Lysine, about 300 mg of Proline, and about 300 mg of KVP as Lys-Pro-Val.

18. The method of claim 15, wherein the immune modulation blend comprises about 750 mg of Ascorbic Acid, about 750 mg of Glutamine (L), about 0.1 mg of Mixed Tocopherols, about 100 mg of Glycine, about 0.1 mg of Vitamin K2 as menaquinone 7, about 0.025 iu of Selenomethionine, about 2.0 mg of PQQ, about 2000 iu of Cholecalciferol, about 100 iu of Retinyl Palmitate, and about 300 mcg of KVP as Lys-Pro-Val.

19. The method of claim 15, wherein the detox blend comprises about 350 mg of Carrot Root, about 200 iu of Retinyl Palmitate, about 350 mg of Calcium d-glucarate, about 150 mg of Leucine, about 300 mg of Molybdenum chelate, about 0.3 mg of Methylfolate, and about 0.15 mg of Methylcobalamin.

20. The method of claim 15, further comprising:
supporting the growth of scar tissue around the silicone implant.
